(19) United States
(12) Patent Application Publication
(10) Pub. No.: US 2008/0137877 A1
(43) Pub. Date: Jun. 12, 2008

(54) Subject actuated system and method for simulating normal and abnormal medical conditions

(75) Inventors: Thomas W. Hubbard, Norfolk, VA (US); Frederic D. McKenzie, Virginia Beach, VA (US); Hector M. Garcia, Norfolk, VA (US); John A. Ullian, Norfolk, VA (US); Gayle A. Gliva-McConvey, Virginia Beach, VA (US); Bo Sun, Norfolk, VA (US)

Correspondence Address: WILMERHALE/DC, 1875 PENNSYLVANIA AVE., NW, WASHINGTON, DC, 20004, US

(73) Assignees: Eastern Virginia Medical School, Norfolk, VA (US); Old Dominion University, Norfolk, VA (US)

(21) Appl. No.: 11/589,912

(22) Filed: Oct. 31, 2006

(51) Int. Cl. A61B 7/04

(52) U.S. Cl. 381/67

(57) ABSTRACT

The present invention provides a system and method for simulating medical conditions to facilitate medical training, that utilizes a stethoscope comprising an earpiece and a headpiece and configured to transmit information indicative of a medical condition, and a triggering device configured to activate the transmission of the information.

### STATEMENT AS TO RIGHTS TO INVENTIONS MADE UNDER FEDERALLY SPONSORED RESEARCH AND DEVELOPMENT

The invention is supported in part by the National Board of Medical Examiners' Stemmler Research Fund Grant number 67-0405. The Government may have certain rights in the invention.

### FIELD OF THE INVENTION

The invention is directed generally to simulating normal and abnormal medical conditions and, more particularly, to transmitting an indication of the medical condition(s) to a recipient to aid in the diagnosis of one or more medical conditions.

### BACKGROUND OF THE INVENTION

To become clinically competent physicians, medical students must develop knowledge and skills in many areas of both the art and science of medicine. Three areas are emphasized in medical students' early clinical training: doctor-patient communication, eliciting the patient history, and performing the physical exam. Standardized patients (SPs), individuals trained to realistically portray patients, are commonly employed to teach and assess medical students in those three areas. By working with SPs, students gain the opportunity to learn and practice the skills of doctor-patient communication, such as eliciting the patient history, conducting the physical exams, and other clinical skills in a safe setting. SPs also provide a way to reliably test students' clinical skills in a realistic setting, interacting with a person. The range of clinical problems an SP can portray, however, is limited. They are typically healthy individuals with few or no abnormal physical conditions. While some can be trained to simulate physical abnormalities (e.g., breathing through one lung, voluntarily increasing blood pressure, etc.), there are many abnormalities that cannot be simulated easily or at all.

One way to supplement what students learn from SPs is for the students to separately learn from and practice on simulators. A variety of mechanical or computer-based simulators are now used in medical education, including software for testing clinical reasoning and diagnostic skills, computer simulations of physiological processes, and physical models for practicing selected procedural skills. For example, a completely virtual SP (e.g., an interactive computer program) has been tried before by Hubal et al., as described in “The Virtual Standardized Patient,” Medicine Meets Virtual Reality 2000 (J. D. Westwood et al., eds., IOS Press), who utilized natural language processing and virtual patients that exhibit emotion in a realistic context to provide completely automatic yet unscripted training sessions. A key limitation to these simulators is that their users (e.g., medical students) do not interact with a live person (a patient or SP). Human-computer interaction brings a different set of psychological concerns than does the human-human interaction of a doctor-patient examination. A significant level of immersion is needed to overcome the human-computer interaction aspects so that there is appreciable transfer of training with regard to patient interaction and diagnosis. This level of immersion and interactivity has not been reached and may not be achievable in a totally virtual form with today's technology. Augmenting SPs with the ability to simulate abnormal physical findings would expand the opportunities for students to learn more clinical skills in a realistic setting with a real person (SP) while practicing their doctor-patient communication skills.

In addition, there is currently a need for expanding the breadth of indications associated with known medical conditions that may be portrayed by an SP. For example, with a real or standardized patient, a student is limited to hearing only the sounds of that single person. Learning a variety of sounds has traditionally required examining many patients over time, often without direct supervision and feedback. Commercially available recordings of heart and lung sounds exist, but using them ignores the process of locating the sources of sounds (e.g., correct placement of the stethoscope) and excludes simultaneous interactions with a patient.

Augmenting SPs with the capability of portraying patients with an increased range of medical conditions would make the use of SPs an even more valuable teaching tool. The present invention is directed to these and other important ends.

### SUMMARY OF THE INVENTION

The invention provides systems and methods for simulating medical conditions to facilitate medical training, that utilizes a stethoscope comprising an earpiece and a headpiece and configured to transmit information indicative of a medical condition, and a triggering device configured to activate the transmission of the information. In some embodiments, a subject is trained to activate the transmission of information using the triggering device when the location of the stethoscope coincides with a predetermined set of regions.

In accordance with the invention, the subject is trained to trigger the transmission of information indicative of a medical condition when the location of the stethoscope coincides with a predetermined set of regions. Such regions include positions over the subject's actual heart, lungs, carotid and renal arteries, aorta, and abdomen. Non-limiting examples of medical conditions that may be simulated using the invention include: bronchitis, heart failure, lung consolidation, pneumonia, atelectasis, pleural effusion, pneumothorax, chronic obstructive pulmonary disease, emphysema, asthma, healthy lung function, mitral valve prolapse, mitral regurgitation, mitral stenosis, pulmonic stenosis, aortic stenosis, aortic regurgitation, ventricular septal defect, pericarditis, healthy heart function, bowel obstruction, renal artery bruits, normal abdominal function, and carotid artery bruits.

In further embodiments, the information indicative of a medical condition is a sound transmitted through the earpiece of the stethoscope. The sound is either a naturally occurring sound or a digitally processed or altered sound. Examples of sounds heard through the stethoscope in accordance with the invention include normal breath sounds, crackles, wheezes, stridor, pleural rub, normal heart sounds, pathologic splitting, murmurs, clicks, gallops, pericardial friction rub, venous hum, bowel sounds, and bruits.

The invention also provides a stethoscope comprising an earpiece and a headpiece and configured to transmit sound indicative of a medical condition, wherein the transmission of sound is activated by a triggering device. In some embodiments, the triggering device uses short-range radiofrequency signals to activate the transmission of sound.

### BRIEF DESCRIPTION OF THE DRAWINGS

FIG. 1 is a system diagram in accordance with an exemplary embodiment of the invention.

FIG. 2 illustrates a stethoscope in accordance with an exemplary embodiment of the invention.

FIG. 3 is a system diagram in accordance with an exemplary embodiment of the invention.

### DETAILED DESCRIPTION OF THE INVENTION

For the purposes of promoting an understanding of the principles of the invention, reference will now be made to specific embodiments and specific language will be used to describe the same. It will nevertheless be understood that no limitation of the scope of the invention is thereby intended, such alteration and further modifications of the invention, and such further applications of the principles of the invention as illustrated herein, being contemplated as would normally occur to one skilled in the art to which the invention relates. Also, it is to be understood that the phraseology and terminology employed herein are for the purpose of description and should not be regarded as limiting.

The invention provides systems and methods for simulating normal and abnormal medical conditions in a subject. In some embodiments, the subject is trained to recognize specific “hot zones” when a user places the head of a modified stethoscope on the subject's body. When the stethoscope head is placed on a predetermined “hot zone” location, the subject actuates a device that triggers the transmission of a selected sound through the earpiece of the stethoscope, or blocks the transmission of a normally occurring sound through the earpiece.

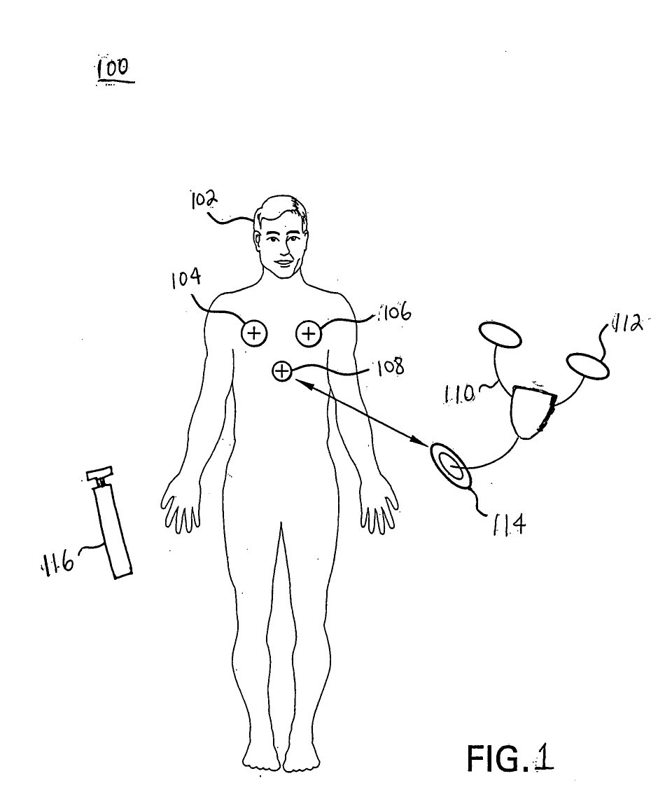

FIG. 1, generally at 100, is a system diagram of an exemplary embodiment of the invention. A user examining subject 102 with stethoscope 110 receives a transmission of a selected sound through earpiece 112 that simulates a medical condition, when stethoscope head 114 is located at a position or a region (“hot zone”) associated with the medical condition (e.g., position 108 in FIG. 1). Hence, the correct placement of stethoscope head 114 on subject 102 correlates with the generation of feedback indicative of a known medical condition to the user. Subject 102 may be, for example, a normal patient or a standardized patient. As used herein, a standardized patient (SP) refers to an individual trained to, or who otherwise can, realistically portray a patient.

As used herein, a “hot zone” refers to a location that the subject is trained to recognize for triggering the playback of a corresponding sound file. “Hot zone” locations on a subject correspond with one or more simulated medical conditions. For example, if a particular cardiovascular condition is desired to be simulated, one or more “hot zones” corresponding to the particular heart condition will be located, for example, over the subject's anterior chest and/or major arteries, such as the carotid or renal artery. As another example, if a particular lung condition is desired to be simulated, one or more “hot zones” corresponding to the particular lung condition will be located, for example, over the subject's lungs. Other “hot zones” in accordance with the invention include areas generally examined by physicians during the pulmonary examination, such as the 26 areas described in Bates' Guide to Physical Examination and History Taking (Bickley & Szilagyi, Philadelphia: Lippincott William & Wilkons 2003), incorporated by reference in its entirety.

The sound files selected for playback into the stethoscope correspond to sounds commonly auscultated during the physical examination of a patient. The auscultation of sounds associated with either a normal or an abnormal medical condition is used, for example, by a user to make a medical diagnosis regarding the medical condition of the subject. As used herein, “auscultation” refers to the act of listening for sounds including those made by internal organs, such as the heart, lungs, abdomen, and major arteries, to aid in the medical diagnosis of a subject.

In accordance with the invention, the auscultated sound corresponds to the particular medical condition that is simulated by the subject. In one embodiment, a heart condition is simulated by the playback of heart-associated sounds. Examples of heart-associated sounds that may be generated, and the associated heart condition that is simulated, include: normal heart sounds, pathologic splitting, murmurs, clicks, gallops, pericardial friction rub, venous hum, and carotid artery bruits, for simulating healthy heart function, heart failure, mitral valve prolapse, mitral regurgitation, mitral stenosis, pulmonic stenosis, aortic stenosis, aortic regurgitation, ventricular septal defect, and pericarditis. In another embodiment, a lung condition is simulated by the generation of lung-associated sounds. Examples of lung-associated sounds that may be generated, and the associated lung condition that is simulated, include: normal breath sounds, crackles, wheezes, stridor, and pleural rub, for simulating healthy lung function, bronchitis, lung consolidation, pneumonia, atelectasis, pleural effusion, pneumothorax, chronic obstructive pulmonary disease, emphysema, and asthma. In another embodiment, an abdominal condition is simulated by the generation of abdominal-associated sounds. Examples of auscultation sounds that may be generated, and the associated abdominal condition that is simulated, include: bowel sounds and bruits, for simulating bowel obstruction, renal or aortic artery bruits, and normal abdominal function.

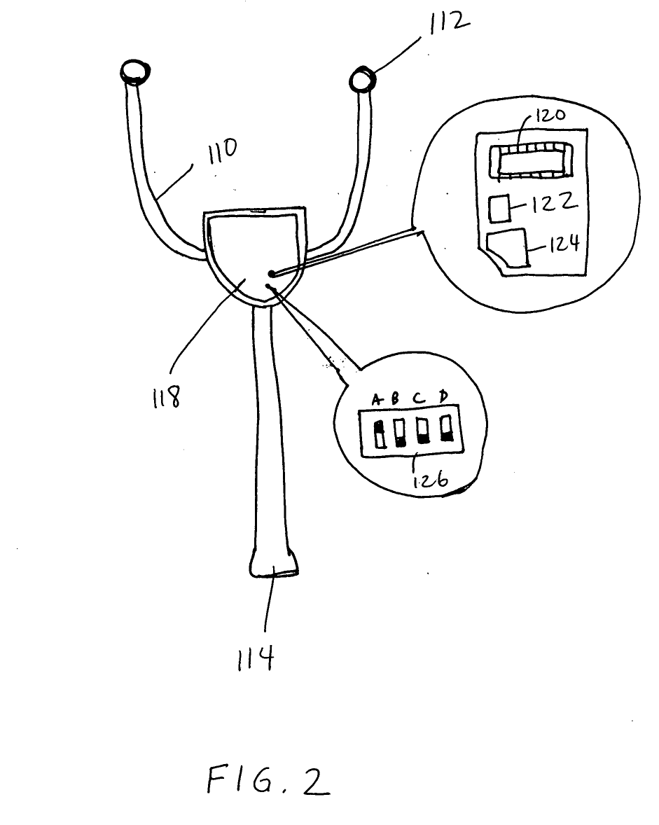

In one or more embodiments of the invention, stethoscope 110 is a standard electronic stethoscope, such as, generally the Androscope i-stethos model IS-28A00 (Andromed, Inc., St. Laurent, Quebec), modified as described herein. FIG. 2 shows an exemplary stethoscope in accordance with the invention. As shown in FIG. 2, stethoscope 110 is modified to contain integrated circuit board 118, on which is mounted sound chip 120, audio output port 122, communications device 124, and selection switch 126.

Sound chip 120 can be programmed through a computer via its serial port to contain one or more sound files. For example, sound chip 120 can be programmed to contain four sound files: A, B, C, and D. Each sound is associated with a known medical condition. For example, A may be the sound of carotid bruits and associated with, for example, atherosclerosis; B may be the sound of end-inspiratory crackles and associated with, for example, congestive heart failure or pneumonia; C may be the sound of end-expiratory wheeze and associated with, for example, asthma; and D may be the sound of borborygmi and associated with, for example, normal abdominal bowel sounds. Sound chip 120 can be a standard, commercially available chip, such as ISD25XXX (Winbond USA) Chip Corder Sound Chip.

Integrated circuit board 118 is also mounted with communications device 124. In operation, communications device 124 signals sound chip 120 to transmit the playback of a selected sound from audio output port 122 to earpiece 112. It is also contemplated that the selected sound can be transmitted directly to earpiece 112, without the use of output port 122. Communications device 124 signals sound chip 120 after it has been signaled by triggering device 116. In other words, a user is not able to detect through earpiece 112 the playback of the selected sound unless communications device 124 has been signaled. If communications device 124 is not signaled, the sound is not detected through earpiece 112. Communications device 124 can be a standard, commercially available device, such as one that uses Bluetooth, RF (radiofrequency), and the like.

Actuation of communications device 124 via a signal can be performed with triggering device 116. Triggering device 116 can be a standard, commercially available device, such as one that uses Bluetooth, RF, and the like. Triggering device 116 is connected to communications device 124 via communication links. Communication links can be either with a cable, such as a serial cable, USB connection, or standard telephone wire connection, or wireless, such as Bluetooth, or any other known short distance wireless communication technology. As shown in FIG. 1, communications device 124 is in communication with triggering device 116 via short-range wireless signals.

The selection of which sound to play from the various sounds stored in the sound banks in sound chip 120 can be performed by the use of selection switch 126 built into integrated circuit 118 and connected to sound chip 120. For example, to select sound file A, the dip switch setting corresponding to A is in the “closed” position while the dip switch settings corresponding to the remaining sound files B, C, and D are in the “open” position. Selection switch 126 can be a standard, commercially available selection switch or dip switch, such as a Futurtec DTP4.

In one or more embodiments of the invention, the sound file is a .wav file. However, other sound files, such as .mp3 files, may be used. The sound file may correspond to either a naturally occurring sound (e.g., a normal heartbeat) or to a sound that has been digitally processed (e.g., modifying a normal heartbeat to sound like an abnormal heartbeat) or digitally altered (e.g., adding extra sounds to normal heart sounds to simulate a valvular defect, or adding wheezes to breath sounds to simulate asthma). Commercially available software for digital signal processing may be used in connection with embodiments of the invention. For example, WaveWarp from SoundsLogical (Glasgow, Scotland UK) may be used.

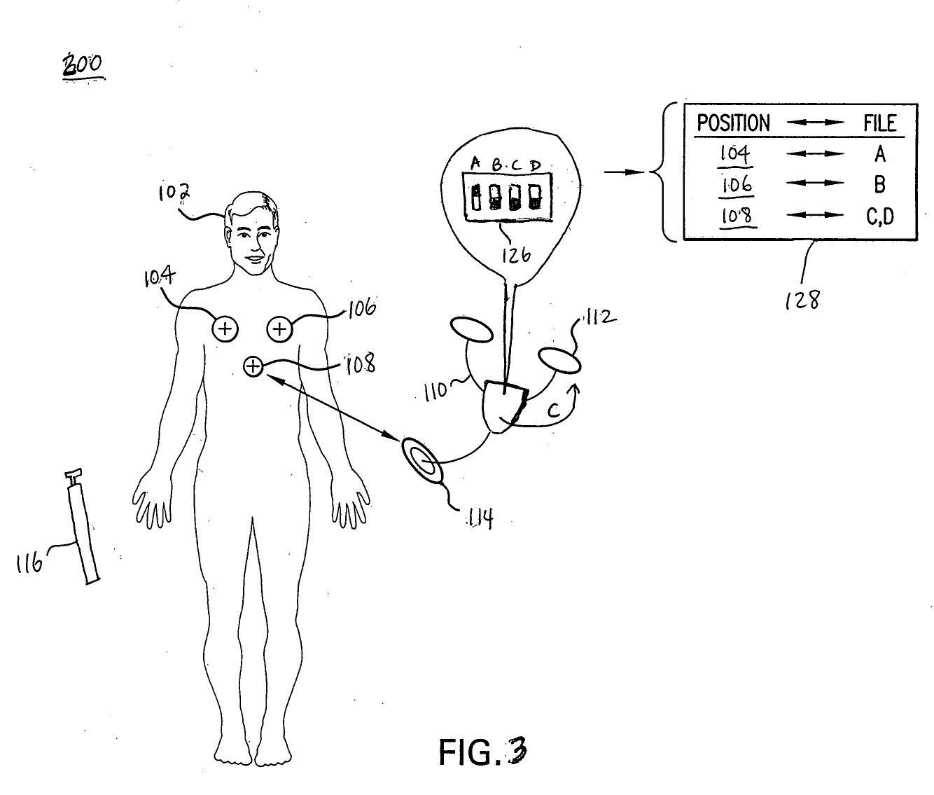

FIG. 3 is a diagram of an exemplary embodiment of the invention. A sound file transmitted to earpiece 112 provides auscultatory feedback to the user when the user places headpiece 114 on an appropriate “hot zone.” Sound file C is selected prior to the examination of subject 102 by moving the dip switch setting corresponding to C in selection switch 126 to the “closed” position and moving the remaining dip switch settings to the “open” position. Subject 102 activates triggering device 116 when the user places headpiece 114 on an appropriate “hot zone.” For example, when the user places headpiece 114 on “hot zone” position 108 over the location of the subject's actual heart, subject 102 activates triggering device 116, which sends a signal to communications device 124, which signals sound chip 120 to play the sounds from the selected sound file C through output 122 and into earpiece 112. The sounds transmitted from sound file C may be abnormal heart sounds, for example associated with a heart defect. Alternatively, sound file D, corresponding to sounds associated with a normal heart, can be selected prior to the examination of subject 102. In this embodiment, when the user places headpiece 114 on “hot zone” position 108 over the subject's actual heart, subject 102 activates triggering device 116, allowing the transmission of sounds from the selected sound file D into earpiece 112. As chart 128 illustrates, various sound files corresponding to different “hot zone” positions can be selected for providing auscultory feedback to the user upon appropriate placement of the stethoscope head.

The invention also contemplates other embodiments wherein the SP can be trained to choose between different sounds as the examination is occurring, rather than requiring the pre-selection of a particular sound prior to the examination. One of skill in the art will recognize that different switches and circuits can be easily modified to allow the playback of different sounds during the examination. One of skill in the art will also recognize that different signals and/or different triggering devices can be used to actuate communications device 124, resulting in the playback of different corresponding sounds. For example, a computer mouse can be used as triggering device 116, so that a right mouse click triggers one sound file and a left mouse click triggers a different sound file.

According to one or more embodiments of the invention, the components of the system for simulating normal or abnormal medical conditions can be connected in different ways. For example, the connections can be wired, wireless, optical, electromagnetic, radio frequency based, and the like. Various components can also be connected across one or more networks so that data can be exchanged and/or analyzed by others. For example, in a classroom setting, an instructor can demonstrate the appropriate locations to position the stethoscope. Appropriate signals can be transmitted across a wireless network. Users in the classroom would then receive the appropriate sounds in their own stethoscopes. All such embodiments, modifications, and variations fall within the scope of the present invention.

While the invention has been illustrated and described in detail in the drawings and foregoing description, the same is to be considered as illustrative and not restrictive in character, it being understood that only the preferred embodiments have been shown and described and that all changes and modifications that come within the spirit of the invention are desired to be protected. In addition, all references cited herein are indicative of the level of skill in the art and are hereby incorporated by reference in their entirety.

We claim:

1. A system for simulating medical conditions to facilitate medical training, the system comprising:
a stethoscope comprising an earpiece and a headpiece and configured to transmit information indicative of a medical condition, and
a triggering device configured to activate the transmission of the information.

2. The system of claim 1, wherein a subject is trained to activate the transmission of the information using the triggering device when the location of the stethoscope coincides with a predetermined set of regions.

3. The system of claim 1, wherein the information indicative of a medical condition is a sound transmitted through the earpiece of the stethoscope.

4. The system of claim 1, wherein the sound is a naturally occurring sound.

5. The system of claim 1, wherein the sound is a digitally processed or altered sound.

6. The system of claim 1, wherein the sound is at least one of: normal breath sounds, crackles, wheezes, stridor, pleural rub, normal heart sounds, pathologic splitting, murmurs, clicks, gallops, pericardial friction rub, venous hum, bowel sounds, and bruits.

7. The system of claim 1, wherein the medical condition is at least one of: bronchitis, heart failure, lung consolidation, pneumonia, atelectasis, pleural effusion, pneumothorax, chronic obstructive pulmonary disease, emphysema, asthma, healthy lung function, mitral valve prolapse, mitral regurgitation, mitral stenosis, pulmonic stenosis, aortic stenosis, aortic regurgitation, ventricular septal defect, pericarditis, healthy heart function, bowel obstruction, renal or aortic artery bruits, normal abdominal function, and carotid artery bruits.

8. The system of claim 2, wherein the predetermined set of regions comprises regions over the heart, lungs, carotid artery, renal artery, and abdomen of a subject being examined.

9. An apparatus configured to transmit sound indicative of a medical condition, wherein the transmission of sound is activated by a triggering device.

10. The apparatus of claim 9, wherein the apparatus is a stethoscope comprising an earpiece and a headpiece.

11. The apparatus of claim 9, wherein the triggering device uses short-range wireless signals to activate the transmission of sound.
